(19) United States
(12) Patent Application Publication
(10) Pub. No.: US 2026/0288200 A1
(43) Pub. Date: Sep. 24, 2026

(54) DETECTION DEVICE

(71) Applicant: Japan Display Inc., Tokyo (JP)

(72) Inventor: Masahiro TADA, Tokyo (JP)

(21) Appl. No.: 19/677,463

(22) Filed: May 14, 2026

Related U.S. Application Data
(63) Continuation of application No. PCT/JP2024/040273, filed on Nov. 13, 2024.

(30) Foreign Application Priority Data
Nov. 20, 2023 (JP) 2023-196876

(51) Int. Cl. G06F 1/16

(52) U.S. Cl. CPC G06F 1/163; G06F 1/1635; G06F 1/1684

(57) ABSTRACT

According to an aspect, a detection device includes: a light source; a plurality of optical sensors; and a control circuit configured to calculate values based on outputs of the optical sensors. The control circuit is configured to perform processing to multiply values corresponding the outputs of the optical sensors by factors individually for each of the values corresponding to the outputs the optical sensors.

### CROSS-REFERENCE TO RELATED APPLICATION

This application claims the benefit of priority from Japanese Patent Application No. 2023-196876 filed on Nov. 20, 2023 and International Patent Application No. PCT/JP2024/040273 filed on Nov. 13, 2024, the entire contents of which are incorporated herein by reference.

### BACKGROUND

### 1. Technical Field

What is disclosed herein relates to a detection device.

### 2. Description of the Related Art

Detection devices that detect a fingertip plethysmogram of a human using a light source that emits light to a finger of the human and a photodetector that receives the light from the light source (for example, Japanese Patent Application Laid-open Publication No. 2022-187539).

An output of an optical sensor can be larger than an output that should be obtained, due to an unintentional occurrence of unwanted components, such as noise. Therefore, with conventional configurations that merely employ the output of one of a plurality of photodetectors that exhibits the highest light-receiving intensity, the output representing the result for emission of the light to the finger of the human is not sufficiently accurate.

For the foregoing reasons, there is a need for a detection device capable of more easily ensuring the accuracy of detection.

### SUMMARY

According to an aspect, a detection device includes: a light source; a plurality of optical sensors; and a control circuit configured to calculate values based on outputs of the optical sensors. The control circuit is configured to perform processing to multiply values corresponding the outputs of the optical sensors by factors individually for each of the values corresponding to the outputs the optical sensors.

### BRIEF DESCRIPTION OF THE DRAWINGS

FIG. 1 is an external view illustrating a detection device according to an embodiment of the present disclosure;

FIG. 2 is a view illustrating an internal structure of the detection device in FIG. 1;

FIG. 3 is a sectional view taken along line X-X′ in FIG. 2;

FIG. 4 is a diagram explaining an exemplary method for using a detection device 100;

FIG. 5 is a block diagram illustrating an internal configuration example of a control circuit 10;

FIG. 6 is a schematic circuit diagram illustrating a configuration related to output systems of electrical signals from optical sensors;

FIG. 7 is a flowchart of a process for deriving vital values;

FIG. 8 is a flowchart of the process included in Step S1;

FIG. 9 is a schematic diagram illustrating a mechanism for obtaining an output of the optical sensor corresponding to pulsation using reflected light of light from a green light-emitting diode (LED);

FIG. 10 is a graph schematically illustrating a method for detecting the pulsation based on the magnitude of the output of the optical sensor;

FIG. 11 is a graph illustrating a relation between a blood oxygen level and a coefficient of absorption of each of red light and near-infrared light by hemoglobin (Hb);

FIG. 12 is a flowchart of a process for deriving the vital values performed in a second embodiment of the present disclosure;

FIG. 13 is a flowchart of a process for deriving the vital values performed in a third embodiment of the present disclosure;

FIG. 14 is a view illustrating an internal structure of the detection device employed in a fourth embodiment of the present disclosure;

FIG. 15 is a flowchart of a process for deriving the vital values performed in the fourth embodiment;

FIG. 16 is a flowchart of a process for deriving the vital values performed in a first modification of the fourth embodiment;

FIG. 17 illustrates diagrams illustrating a concept of a difference between a vital value V1 and a vital value V2;

FIG. 18 is a flowchart of a process for deriving the vital values performed in a second modification of the fourth embodiment; and

FIG. 19 illustrates diagrams illustrating the concept of a difference between a difference value N5 and a difference value N6.

### DETAILED DESCRIPTION

The following describes embodiments of the present disclosure with reference to the drawings. What is disclosed herein is merely an example, and the present disclosure naturally encompasses appropriate modifications easily conceivable by those skilled in the art while maintaining the gist of the present invention. To further clarify the description, the drawings may schematically illustrate, for example, widths, thicknesses, and shapes of various parts as compared with actual aspects thereof. However, they are merely examples, and interpretation of the present disclosure is not limited thereto. The same element as that described with reference to an already mentioned drawing is denoted by the same reference numeral through the present specification and the drawings, and detailed description thereof may not be repeated where appropriate.

### First Embodiment

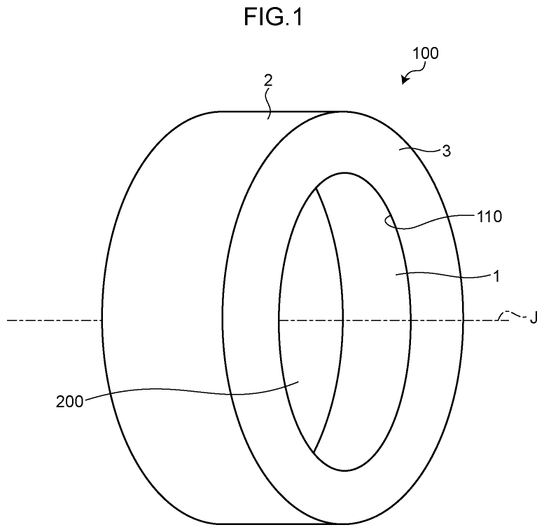

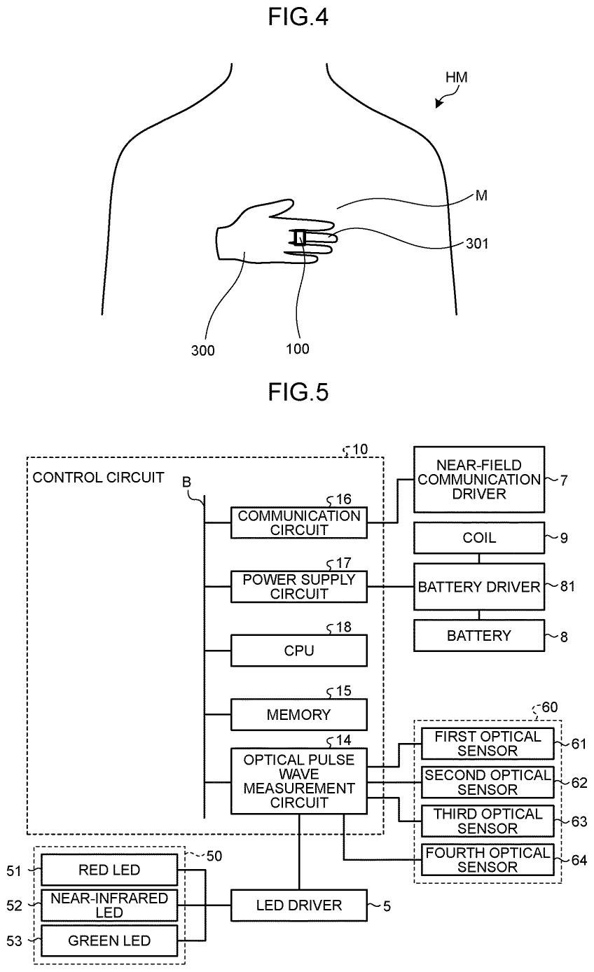

FIG. 1 is an external view illustrating a detection device according to a first embodiment of the present disclosure. In FIG. 1, a detection device 100 has a shape of a finger ring. The detection device 100 has a hollow portion 200. A finger can be inserted into the hollow portion 200 of the detection device 100. That is, a user of the detection device 100 can wear the detection device 100 on one finger (for example, a finger 301 of a human HM in FIG. 4).

In FIG. 1, the detection device 100 has an inner surface portion 1, an outer surface portion 2, and a side surface portion 3. As illustrated in FIG. 4 to be explained later, the inner surface portion 1 contacts the finger while the detection device 100 is worn on the finger 301. That is, the inner surface portion 1 has an inner surface 110 that contacts the finger when the detection device 100 is worn on the finger. The inside of the inner surface 110 is the hollow portion 200 into which the finger 301 can be inserted. The outer surface portion 2 is provided outside the inner surface portion 1. When a center line J denotes a line passing through the center of the hollow portion 200, the inner surface portion 1 and the outer surface portion 2 are cylindrical members with the center line J serving as the central axis. The side surface portion 3 is a plate-like member along a plane intersecting (for example, orthogonal to) the center line J, and is an annular member centered on the center line J. The side surface portion 3 is not required to be completely orthogonal to the center line J, and only needs to be provided such that the inner surface portion 1, the outer surface portion 2, and the side surface portions 3 form the housing of the detection device 100.

Hereinafter, the term “diameter” or “radius” refers to the diameter or the radius of a circle centered on the center line J unless otherwise noted. The term “radial direction” refers to a direction along the radius of a circle centered on the center line J.

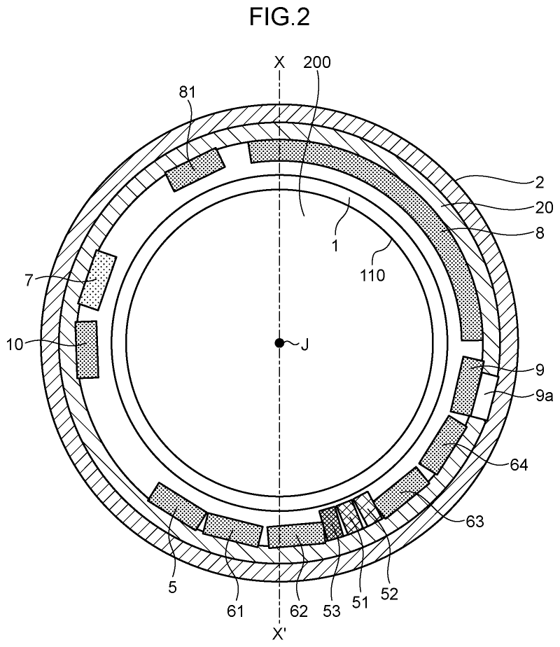

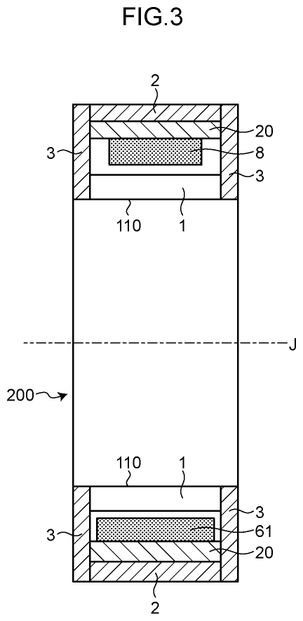

FIG. 2 is a view illustrating an internal structure of the detection device 100 in FIG. 1. FIG. 2 is a view illustrating a section obtained by cutting the detection device 100 in FIG. 1 along a plane orthogonal to the center line J. FIG. 2 illustrates a section obtained by cutting the detection device 100 along a plane passing through the midpoint in the longitudinal direction of the outer surface portion 2 extending along the center line J. FIG. 3 is a sectional view taken along line X-X′ in FIG. 2.

The diameter of the outer surface portion 2 centered on the center line J is longer than the diameter of the inner surface portion 1 centered on the center line J. The inner diameter of the ring of the side surface portion 3 corresponds to the diameter of the inner surface portion 1. The outer diameter of the ring of the side surface portion 3 corresponds to the diameter of the outer surface portion 2. As illustrated in FIG. 3 to be explained later, two side surface portions 3 are provided facing each other with the inner surface portion 1 and the outer surface portion 2 interposed therebetween. The side surface portions 3 are joined to the inner surface portion 1 on the inner peripheral side and to the outer surface portion 2 on the outer peripheral side. The inner surface portion 1, the outer surface portion 2, and the two side surface portions 3 are combined in this way to form the outer shape of the detection device 100 into a ring shape, as illustrated in FIG. 1. That is, the inner surface portion 1, the outer surface portion 2, and the two side surface portions 3 form a housing of the ring-shaped detection device 100.

In FIG. 2, the detection device 100 includes a light-emitting diode (LED) driver 5, a first optical sensor 61, a second optical sensor 62, a third optical sensor 63, a fourth optical sensor 64, a near-field communication driver 7, a battery 8, a coil 9, a control circuit 10, a red LED 51, a near-infrared LED 52, and a green LED 53. These components are located outside the inner surface portion 1 and inside the outer surface portion 2, that is, between the inner surface portion 1 and the outer surface portion 2. These components are provided on a flexible substrate 20. These components can mutually exchange signals via the flexible substrate 20.

Hereinafter, the term “light source 50″ collectively refers to the red LED 51, the near-infrared LED 52, and the green LED 53. The term ”optical sensor 60″ collectively refers to the first optical sensor 61, the second optical sensor 62, the third optical sensor 63, and the fourth optical sensor 64 (refer to FIG. 5).

The flexible substrate 20 has an annular shape centered on the center line J. The flexible substrate 20 is provided along the inner peripheral surface of the outer surface portion 2. The radial positional relation between the inner surface portion 1, the flexible substrate 20, and the outer surface portion 2 is in the following order: the inner surface portion 1, the flexible substrate 20, and the outer surface portion 2 from the center side toward the outside with the center line J as the center. The components provided on the flexible substrate 20 are located radially outside the inner surface portion 1 and radially inside the flexible substrate 20.

The red LED 51 outputs red light. The near-infrared LED 52 outputs near-infrared light. The green LED 53 outputs green light. That is, the detection device 100 includes the light sources provided outside the inner surface portion 1 and inside the outer surface portion 2 (between the inner surface portion 1 and the outer surface portion 2). The LED driver 5 drives the red LED 51, the near-infrared LED 52, and the green LED 53 to emit light.

The red LED 51 is a light source that emits red light. The near-infrared LED 52 is a light source that emits near-infrared light. The green LED 53 is a light source that emits green light. The optical axes of the light emitted from the red LED 51, the light emitted from the near-infrared LED 52, and the light emitted from the green LED 53 each extend toward the center line J. The inner surface portion 1 is a light-transmitting member. Thus, the light emitted from the light source 50 (the red LED 51, the near-infrared LED 52, and the green LED 53) can pass through the inner surface portion 1. Therefore, when the finger 301 is in the hollow portion 200, the light emitted from the light source 50 (the red LED 51, the near-infrared LED 52, and the green LED 53) is reflected and scattered by the finger 301. The light thus reflected and scattered can be detected by the optical sensor 60. In other words, the optical sensor 60 is configured to detect the light that has been emitted from the light source 50 (the red LED 51, the near-infrared LED 52, and the green LED 53) and then reflected and scattered. The outer surface portion 2 and the side surface portions 3 are light-blocking members.

To give a specific example, the inner surface portion 1 of the first embodiment is made of a light-transmitting resin. The outer surface portion 2 and the side surface portions 3 are made of a non-light-transmitting metal, a non-light-transmitting alloy, a non-light-transmitting metallic compound, or a metallic compound resin. The compositions of the inner surface portion 1, the outer surface portion 2, and the side surface portions 3 are not limited to these materials. Other materials with suitable light transmittance and suitable strength for forming a housing may be used.

The optical sensor 60 receives the light that has been output from the light source 50 (the red LED 51, the near-infrared LED 52, and the green LED 53) and transmitted through the finger in the hollow portion 200. The optical sensor 60 detects changes in intensity of the received light. The optical sensor 60 is configured with organic photodiodes (OPDs), for example, and outputs electrical signals corresponding to the light emitted thereto. That is, in the first embodiment, the first optical sensor 61, the second optical sensor 62, the third optical sensor 63, and the fourth optical sensor 64 are each an OPD. The optical sensor 60 may be configured with optical sensors based on other methods than that of OPDs.

The following describes the positional relation between the light source 50 and the optical sensor 60 in the circumferential direction along the inner peripheral surface of the flexible substrate 20. Two of the first optical sensor 61, the second optical sensor 62, the third optical sensor 63, and the fourth optical sensor 64 are positioned on one side of the light source 50 in the circumferential direction, and the other two are positioned on the other side of the light source 50 in the circumferential direction. In the first embodiment, as illustrated in FIG. 2, the first optical sensor 61, the second optical sensor 62, the green LED 53, the red LED 51, the near-infrared LED 52, the third optical sensor 63, and the fourth optical sensor 64 are arranged in this order from one side toward the other side in the circumferential direction. The direction from one side toward the other side in the circumferential direction refers herein to the counterclockwise direction in FIG. 2, and is a direction starting from the first optical sensor 61.

In the first embodiment, when one of the red LED 51, the near-infrared LED 52, or the green LED 53 is on, the other two are not on. When the red LED 51 is on and the near-infrared LED 52 and the green LED 53 are not on, the red light transmitted through the finger is received by the optical sensor 60. When the near-infrared LED 52 is on and the red LED 51 and the green LED 53 are not on, the near-infrared light transmitted through the finger is received by the optical sensor 60. When the green LED 53 is on and the red LED 51 and the near-infrared LED 52 are not on, the green light transmitted through the finger is received by the optical sensor 60.

The near-field communication driver 7 includes an antenna, which is not illustrated. The near-field communication driver 7 exchanges signals between the detection device 100 and other devices. The near-field communication driver 7 can transmit data and the like measured by the detection device 100 to the other devices. The near-field communication driver 7 can also receive data and the like transmitted by the other devices.

The battery 8 supplies power to the various parts of the detection device 100. The battery 8 is a lithium-ion battery, for example. A battery driver 81 controls the battery 8. The battery 8 is charged by the battery driver 81.

The coil 9 is a coil for charging the battery 8. The coil 9 includes windings wound along the outer periphery of the inner surface portion 1 on the outer side of the inner surface portion 1 and the inner side of the outer surface portion 2 (between the inner surface portion 1 and the outer surface portion 2). As will be described later, an induced current based on an applied magnetic field flows through the coil 9. The coil 9 includes a coil 91 and a coil 92 (not illustrated). The coils 91 and 92 are each wound along the outer peripheral surface of the inner surface portion 1. The coil 91 is provided on one end side of the outer surface portion 2 along the center line J, and the coil 92 is provided on the other end side of the outer surface portion 2 along the center line J. The coils 91 and 92 are coupled in parallel to the battery driver 81. This configuration allows the battery 8 to be charged by the induced current flowing through either one of the coils 91 and 92. The windings of the coils 91 and 92 are each coated to maintain insulation. A portion of an area located radially outside the coil 9 is a notch 9a not provided with the flexible substrate 20. This configuration makes wireless charging by the coil 9 more efficient.

The control circuit 10 controls the various parts of the detection device 100. The control circuit 10 is an integrated circuit (IC), such as a microcontroller. The control circuit 10 may be, for example, a programmable logic device (PLD) such as a field-programmable gate array (FPGA).

FIG. 4 is a diagram explaining an exemplary method for using the detection device 100. As illustrated in FIG. 4, the detection device 100 is worn on the finger 301 of a hand 300 of the human HM. The detection device 100 performs various operations to derive vital values of the human HM.

FIG. 5 is a block diagram illustrating an internal configuration example of the control circuit 10. As illustrated in FIG. 5, the control circuit 10 of this example includes an optical pulse wave measurement circuit 14, a memory 15, a communication circuit 16, a power supply circuit 17, and a central processing unit (CPU) 18. These components are coupled by a bus B and can exchange data with one another via the bus B.

The optical pulse wave measurement circuit 14 is coupled to the LED driver 5 and the optical sensor 60. The optical pulse wave measurement circuit 14 amplifies and digitizes analog outputs from the optical sensor 60 and stores them in the memory 15.

The memory 15 is a storage that stores therein various types of data. The memory 15 may include, for example, a random-access memory (RAM), a read-only memory (ROM), an electrically erasable programmable read-only memory (EEPROM), and the like.

The communication circuit 16 is coupled to the near-field communication driver 7. The communication circuit 16 transmits measurement results and so forth to an external device. The external device is, for example, a portable electronic apparatus, such as a smartphone or a tablet device, held by the user of the detection device 100. The portable electronic apparatus, such as the smartphone or the tablet device, includes a display screen. By displaying data from the detection device 100 on the screen, the user of the detection device 100 can check the data received from the detection device 100.

The power supply circuit 17 is coupled to the battery driver 81. The power supply circuit 17 controls the charging of the battery 8 and supplies the power from the battery 8 to the various parts.

The CPU 18 is a controller that controls various parts in the control circuit 10. The CPU 18 executes predetermined computer programs to measure or calculate biometric information, such as a pulse wave velocity, blood pressure, and the pulse frequency.

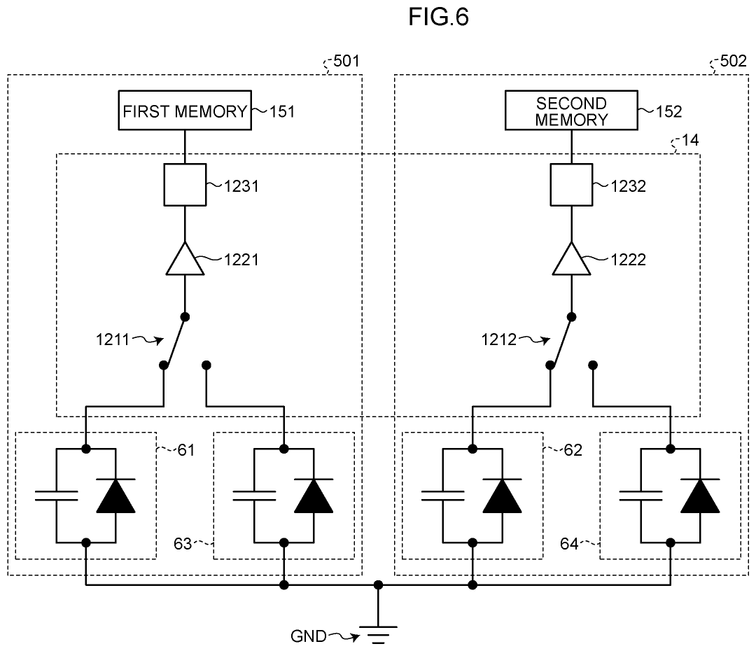

FIG. 6 is a schematic circuit diagram illustrating a configuration related to output systems of the electrical signals from the optical sensor 60. In the first embodiment, a first system 501 and a second system 502 are provided. As illustrated in FIG. 6, the first optical sensor 61 and the third optical sensor 63 are included in the first system 501. The second optical sensor 62 and the fourth optical sensor 64 are included in the second system 502.

The first system 501 includes a switch 1211, an amplifier 1221, a converter 1231, and a first memory 151. The second system 502 includes a switch 1212, an amplifier 1222, a converter 1232, and a second memory 152.

Each of the switches 1211 and 1212 is a switch that couples one of the two optical sensors to the amplifying circuit and decouples the other from the amplifying circuit. Each of the switches 1211 and 1212 is capable of switching between the one optical sensor and the other optical sensor. For example, the switch 1211 is capable of switching between a state in which an output from the first optical sensor 61 is transmitted to the amplifier 1221 and a state in which an output from the third optical sensor 63 is transmitted to the amplifier 1221. The switch 1212 is capable of switching between a state in which an output from the second optical sensor 62 is transmitted to the amplifier 1222 and a state in which an output from the fourth optical sensor 64 is transmitted to the amplifier 1222.

The amplifiers 1221 and 1222 are circuits that serve as amplifiers (AMPs) that amplify the outputs transmitted from the optical sensors. The converters 1231 and 1232 are analog-to-digital conversion circuits (analog-to-digital converters (ADCs)) that convert analog signals amplified by the amplifying circuits into digital signals. The first and the second memories 151 and 152 are memory circuits that store therein the digital signals output from the ADCs.

In the first embodiment, a “value corresponding to the output of the optical sensor” refers to a value obtained by amplifying the output of the optical sensor with the amplifier and digitalizing the amplified output with the analog-to-digital conversion circuit. The optical sensor is, for example, the first optical sensor 61, the second optical sensor 62, the third optical sensor 63, or the fourth optical sensor 64. The amplifier is, for example, the amplifier 1221 or 1222. The analog-to-digital conversion circuit is, for example, the converter 1231 or 1232. That is, the magnitude of the digitized value indicates the intensity of the output of the optical sensor. The amplification of the output of the optical sensor using the amplifier 1221 or 1222 is not essential and can be omitted.

The first optical sensor 61 or the third optical sensor 63 is coupled to the amplifier 1221 via the switch 1211. The amplifier 1221 is coupled to the first memory 151 via the converter 1231. The second optical sensor 62 or the fourth optical sensor 64 is coupled to the amplifier 1222 via the switch 1212. The amplifier 1222 is coupled to the second memory 152 via the converter 1232. A configuration (for example, reference potential electrode or the like) that exhibits a reference potential GND is provided and coupled to the first optical sensor 61, the second optical sensor 62, the third optical sensor 63, and the fourth optical sensor 64. Specifically, the first optical sensor 61 and the third optical sensor 63 are provided between the switch 1211 and the reference potential GND, and the second optical sensor 62 and the fourth optical sensor 64 are provided between the switch 1212 and the reference potential GND.

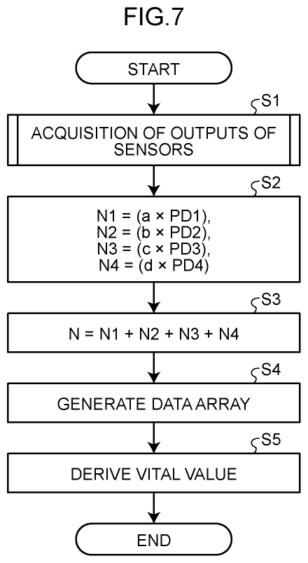

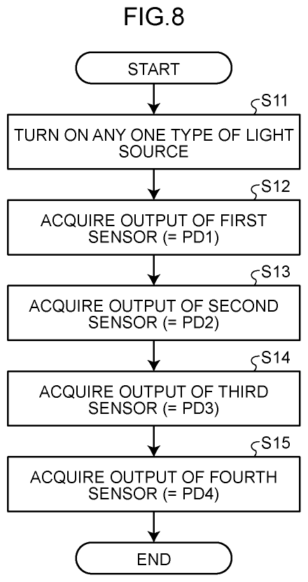

The configuration illustrated in FIG. 6 is included in the control circuit 10. As illustrated in FIG. 5, the control circuit 10 includes the optical pulse wave measurement circuit 14, the memory 15, and other components. The optical pulse wave measurement circuit 14 includes the switches 1211 and 1212, the amplifiers 1221 and 1222, and the converters 1231 and 1232. The first and the second memories 151 and 152 may be provided as a cache memory in the optical pulse wave measurement circuit 14 or provided as a configuration included in the memory 15. In either case, the first and the second memories 151 and 152 are configurations included in the control circuit 10, and data stored in the first and the second memories 151 and 152 is also stored in the memory 15. The digital data generated according to the output of each of the first optical sensor 61, the second optical sensor 62, the third optical sensor 63, and the fourth optical sensor 64 is stored in the memory 15 and referenced in the processing by the CPU 18. The CPU 18 performs various processes related to the derivation of the vital values. The following describes a process for deriving the vital values with reference to FIGS. 7 and 8.

FIG. 7 is a flowchart of a process for deriving the vital values. First, the outputs of the sensors are acquired (Step S1).

FIG. 8 is a flowchart of a process included in Step S1. Step S1 includes Step S11, Step S12, Step S13, Step S14, and Step S15. Steps S12, S13, S14, and S15 are performed in no particular order. Steps S12, S13, S14, and S15 each include the digitizing processing by the optical pulse wave measurement circuit 14 described above.

The processing at Step S11 turns on any of the following light sources: the red LED 51, the near-infrared LED 52, and the green LED 53. Specifically, the CPU 18 outputs a command to turn on any one of the red LED 51, the near-infrared LED 52, and the green LED 53 to the light source 50. The light source 50 turns on any one of the red LED 51, the near-infrared LED 52, and the green LED 53 in response to the command. The processing at Step S12 acquires the output of the first optical sensor 61 as an output PD1. The processing at Step S13 acquires the output of the second optical sensor 62 as an output PD2. The processing at Step S14 acquires the output of the third optical sensor 63 as an output PD3. The processing at Step S15 acquires the output of the fourth optical sensor 64 as an output PD4. The output from each of the first optical sensor 61, the second optical sensor 62, the third optical sensor 63, and the fourth optical sensor 64 is digitized via the circuit configuration described with reference to FIG. 6, whereby the output becomes data that can be referenced by the CPU 18. The CPU 18 performs the processing at Steps S12, S13, S14, and S15 to acquire the data. Each of the outputs PD1, PD2, PD3, and PD4 is a value that indicates the intensity of the output.

After the processing at Step S1 in FIG. 7, the CPU 18 calculates values N1, N2, N3, and N4 (Step S2). N1 is a value obtained by multiplying the output PD1 by a weighting factor a. N2 is a value obtained by multiplying the output PD2 by a weighting factor b. N3 is a value obtained by multiplying the output PD3 by a weighting factor c. N4 is a value obtained by multiplying the output PD4 by a weighting factor d.

Thus, the control circuit 10 including the CPU 18 performs the processing to multiply the values corresponding to the outputs of the optical sensor 60 including a plurality of optical sensors (for example, first optical sensor 61, second optical sensor 62, third optical sensor 63, and fourth optical sensor 64) by factors (for example, a, b, c, and d) individually for the value corresponding to the output of each of the optical sensors. Thus, the control circuit 10 including the optical pulse wave measurement circuit 14 and the CPU 18 serves as a control circuit that calculates values based on the outputs of the optical sensors. In the control circuit 10, the optical pulse wave measurement circuit 14 generates the “values corresponding to the outputs of the optical sensors” as described above. The CPU 18 calculates the values based on the outputs of the optical sensors by performing the arithmetic processing including the processing to multiply the “values corresponding to the outputs of the optical sensors” by the factors. The factors a, b, c, and d are any weighting factors and are set as predetermined real numbers. In the first embodiment, for example, a=b=c=d=1, but the factors are not limited to this value.

After the processing at Step S2, the CPU 18 calculates a measured value N by summing the values N1, N2, N3, and N4 (Step S3).

The CPU 18 generates a data array in which the measured values N obtained by performing Steps S1 to S3 are arranged in chronological order (Step S4). That is, the acquisition of the outputs of the sensors by the processing at Step S1 is performed a plurality of times during a certain period of time (for example, one minute). Steps S2 and S3 are also performed a plurality of times corresponding to the number of operations of the processing at Step S1. In Step S4, the CPU 18 generates the data array in which the measured values N generated in the processing at Step S3 performed a plurality of times are arranged in chronological order. The certain period of time is not limited to one minute and can be changed as appropriate. However, when the certain period of time is one minute, the pulse rate can be expressed in beats per minute (bpm).

The CPU 18 derives the vital values from the data array generated in the processing at Step S4 (Step S5). Examples of the vital values derived in the first embodiment include, but are not limited to, a value indicating the pulsation and a value indicating a blood oxygen level (SpO₂). The following sequentially describes how the vital values are derived.

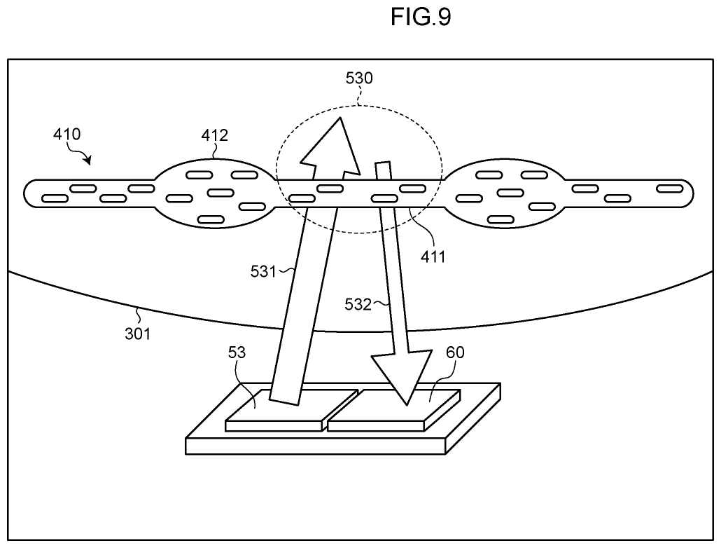

FIG. 9 is a schematic diagram illustrating a mechanism for obtaining the output of the optical sensor 60 corresponding to pulsation using reflected light of the light from the green LED 53. Irradiation light 531 that is the light emitted from the green LED 53 irradiates an irradiation area 530 of the finger 301. An artery 410 in the finger 301 runs in the irradiation area 530, and blood flows through the artery 410. Part of the irradiation light 531 is reflected by biological tissues in the finger 301 including the artery 410 and becomes reflected light 532, and then enters the optical sensor 60. The optical sensor 60 detects the reflected light 532 thus generated.

As illustrated in FIG. 9, a non-dilated portion 411 and a dilated portion 412 of the artery 410 are produced. The non-dilated portion 411 and the dilated portion 412 are produced due to the pumping out of the blood into the artery 410 by the heartbeat. That is, the non-dilated portion 411 and the dilated portion 412 are produced according to the pulsation. In FIG. 9, the artery 410 within the irradiation area 530 is in a non-dilated state (i.e., the non-dilated portion 411), but the artery 410 within the irradiation area 530 may be brought into the dilated state (i.e., the dilated portion 412). The number of times the dilated portion 412 is produced at the same location within a certain period of time is referred to as the “pulse rate” or “heart rate”. The value indicating the pulsation among the vital values mentioned above is the same value as the pulse rate.

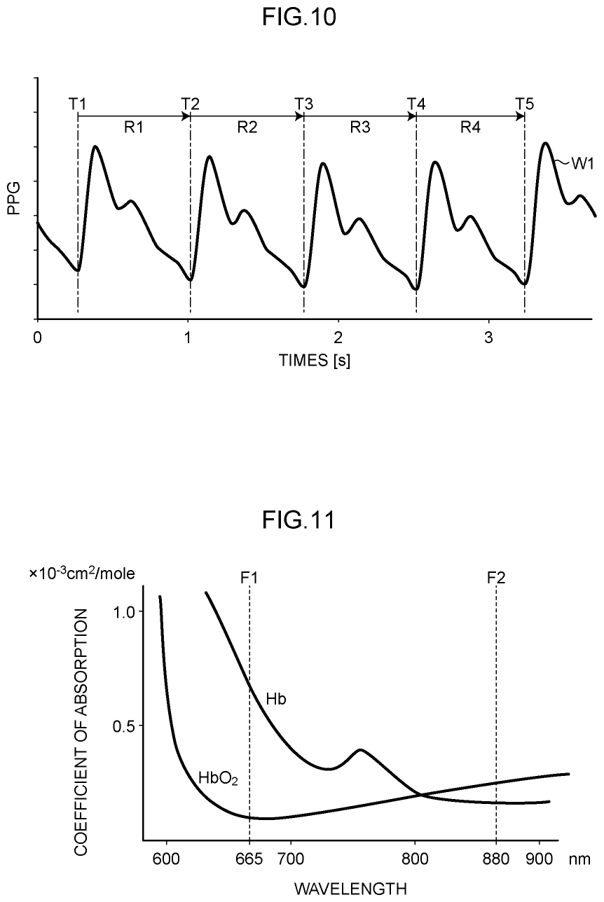

FIG. 10 is a graph schematically illustrating a method for detecting the pulsation based on the magnitude of the output of the optical sensor 60. The vertical axis in FIG. 10 indicates the intensity of the output of the optical sensor 60, and the horizontal axis indicates the lapse of time. In the detection of the pulsation, photoplethysmography (PPG) is used. Even at the same location (for example, irradiation area 530) in the artery 410, the volume of blood flowing through the location differs between the time when the non-dilated portion 411 is produced and the time when the dilated portion 412 is produced. Such a difference in volume of blood is manifested in the degree of generation of the reflected light 532. That is, the output of the optical sensor 60 obtained by continuing the process of the reflected light detection for a certain period of time indicates the number of times of generation of the dilated portion 412 produced during that certain period of time. The reflected light detection denotes the detection, using the optical sensor 60, of the degree of generation of the reflected light 532 produced by irradiating the irradiation area 530 with the irradiation light 531.

Specifically, the non-dilated portion 411 and the dilated portion 412 are cyclically alternately produced in response to the pulsation. Therefore, the output of the optical sensor 60 also exhibits cyclical strong/weak patterns corresponding to the cyclical generation of the non-dilated portion 411 and the dilated portion 412, as illustrated in FIG. 10. Based on the number of generations of a characteristic output that cyclically occur in such a pattern, the number of outputs indicating the number of occurrences of the dilated portion 412, that is the pulse rate, can be identified.

FIG. 10 specifically illustrates timings T1, T2, T3, T4, and T5. At each of timings T1, T2, T3, T4, and T5, a characteristic output occurs. The characteristic output is the lower limit output obtained immediately before the output of the optical sensor 60 rises sharply. At time T1, the artery 410 in the irradiation area 530 is the non-dilated portion 411. After that, the artery 410 in the irradiation area 530 becomes the dilated portion 412 due to the pulsation with the lapse of time illustrated as time R1, and then begins returning to be the non-dilated portion 411. At time T2, the state of the artery 410 returns to the non-dilated portion 411 again. That is, during time R1, the artery 410 pulses once. Thereafter, under the same concept, the artery 410 pulses once during time R2, goes through the state of the dilated portion 412, and at time T3, the state of the artery 410 returns to the non-dilated portion 411. During time R3, the artery 410 pulses once and goes through the state of the dilated portion 412, and at time T4, the state of the artery 410 returns to the non-dilated portion 411. During time R4, the artery 410 pulses once and goes through the state of the dilated portion 412, and at time T5, the state of the artery 410 returns to the non-dilated portion 411. Thus, based on the number of generations of the characteristic output included in the output of the optical sensor 60, the number of outputs indicating the number of occurrences of the dilated portion 412, that is the pulse rate, can be identified.

When obtaining the output of the optical sensor 60 for the purpose of obtaining the pulse rate as in the PPG, light reflected from the artery 410 in the finger 301 can be obtained more easily from the green light than from the red light or the near-infrared light. Therefore, when obtaining the value indicating the pulsation among the vital values, the green light, that is, the light from the green LED 53, is used.

FIG. 11 is a graph illustrating a relation between the blood oxygen level and a coefficient of absorption of each of the red light and the near-infrared light by hemoglobin (Hb). In FIG. 11, the position in the horizontal axis direction where a dashed line F1 is located indicates the coefficient of absorption of the red light from the red LED 51. The position in the horizontal axis where a dashed line F2 is located indicates the coefficient of absorption of the near-infrared light from the near-infrared LED 52.

Red blood cells in blood contain hemoglobin. Hemoglobin has different coefficients of absorption for the red light and the near-infrared light between an oxyhemoglobin (HbO₂) state where hemoglobin is bound to oxygen and a hemoglobin (Hb) state where hemoglobin is not bound to oxygen. Specifically, as illustrated in FIG. 11, hemoglobin has a higher coefficient of absorption for the red light and a lower coefficient of absorption for the near-infrared light than those of oxyhemoglobin. Such properties of hemoglobin and oxyhemoglobin can be used to measure the blood oxygen levels. Specifically, the first intensity of light and the second intensity of light depend on the ratio between hemoglobin and oxyhemoglobin in the blood. The first intensity of light is the intensity of light scattered by blood flowing through the artery 410 in the finger 301 and detected by the optical sensor 60 when the red light from the red LED 51 irradiates the finger 301. The second intensity of light is the intensity of light scattered by the blood and detected by the optical sensor 60 when the near-infrared light from the near-infrared LED 52 irradiates the finger 301. Therefore, the blood oxygen level can be identified by performing at least one of a first process and a second process. The first process is a process to turn on the red LED 51 and detect scattered light using the optical sensor 60, and the second process is a process to turn on the near-infrared LED 52 and detect scattered light using the optical sensor 60. In the first embodiment, both the first and second processes are performed.

As describe with reference to FIG. 10, the pulsation occurs in the artery 410. Therefore, when detecting the red light and the near-infrared light, the detection results vary depending on changes in blood flow rate according to the state of the pulsation at the location irradiated with light, such as in the irradiation area 530. However, the ratio of the degree of detection of the red light to the degree of detection of the near-infrared light within a very small time difference as close to the same time as possible is similar to that represented by the dashed lines F1 and F2 described with reference to FIG. 11. Therefore, the blood oxygen level can be identified without problems. The pulsation can also be derived using the red light and the near-infrared light, based on the detection result variation corresponding to the changes in the blood flow rate. In the first embodiment, the green light is used to identify the pulse rate for the purpose of more accurate identification of the pulse rate.

If a value indicating the pulsation is derived as a vital value in the processing at Step S5, the light source to be turned on in the processing at Step S11 is the green LED 53. If a value indicating the pulsation is derived as a vital value in the processing at Step S5, the light sources to be turned on in the processing at Step S11 is the red LED 51 and the near-infrared LED 52. However, the red LED 51 and the near-infrared LED 52 are not turned on at the same time. The data array of the measured values N obtained when the red LED 51 is on and the data array of the measured values N obtained when the near-infrared LED 52 is on are generated separately.

As described above, according the first embodiment, the detection device (for example, detection device 100) includes the light source (for example, light source 50), the optical sensors (for example, first optical sensor 61, second optical sensor 62, third optical sensor 63, and fourth optical sensor 64), and the control circuit (for example, control circuit 10) that calculates the values based on the outputs of the optical sensors. The control circuit performs the processing to multiply the values corresponding to the outputs of the optical sensors by the factors (for example, a, b, c, and d) individually for the value corresponding to the output of each of the optical sensors. This configuration allows any adjustment of the output corresponding to the degree of detection of light by each of the optical sensors. For example, when there is no particular problem in the degree of detection of light of each of the optical sensors, the weighting factors may be set as a=b=c=d=1, as described above in the first embodiment. When a larger gain should be applied to some of the outputs of the optical sensors, the factors for values corresponding to the some of the outputs can be set larger than 1. When some of the outputs of the optical sensors are excessive, the factors for values corresponding to the some of the outputs can be set smaller than 1. Thus, according to the present embodiment, any adjustment of the output of each of the optical sensors can be made according to the setting of the factors. With the adjustment of the outputs, the saturation of the output and/or the decrease in detection accuracy caused by an excessively weak output can be inhibited. Therefore, the accuracy of detection can be more easily ensured.

Furthermore, in the present embodiment, the values corresponding to the outputs of the optical sensors are individually multiplied by the factors and then summed, and the average of the summed values is calculated. As a result, even if the outputs of some of the optical sensors deviate from the outputs of the other optical sensors, effects of the outputs of the some of the optical sensors can be easily reduced. Therefore, the accuracy of detection can be more easily ensured.

The optical sensor 60 is the OPD, that is, the organic photodiode, which makes the detection device 100 lighter and easier to manufacture at a lower cost than in the case of an inorganic photodiode. The organic photodiodes can be more flexibly arranged and more easily fabricated on a curved surface such as the inner peripheral surface of the flexible substrate 20 than inorganic photodiodes.

Since the light source (for example, light source 50), the optical sensors (for example, first optical sensor 61, second optical sensor 62, third optical sensor 63, and fourth optical sensor 64), and the control circuit (for example, control circuit 10) are accommodated in the finger ring-shaped housing, the detection device 100 can be easily worn on the finger 301 of the human HM.

### Second Embodiment

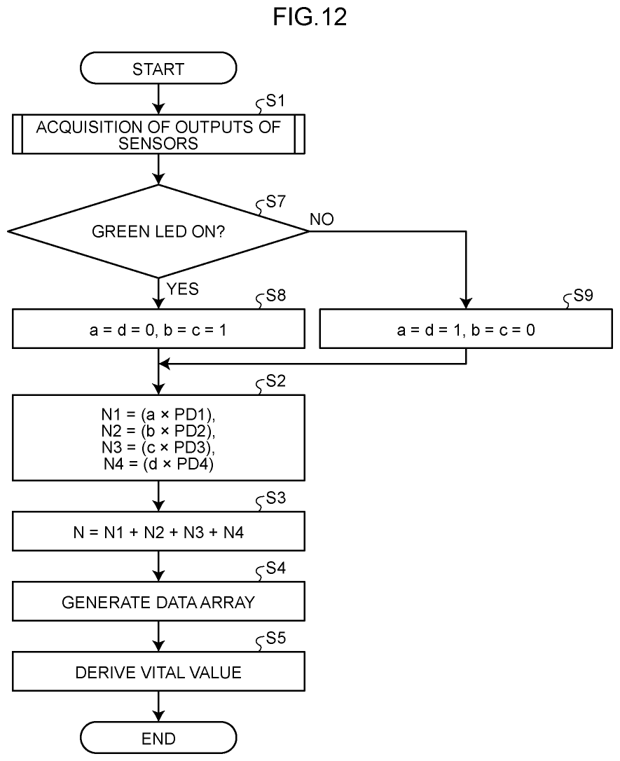

The following describes a second embodiment of the present disclosure in which processing partially different from that of the first embodiment is performed, with reference to FIG. 12.

FIG. 12 is a flowchart of a process for deriving the vital values performed in the second embodiment. In the second embodiment, after the processing at Step S1 described with reference to FIGS. 7 and 8 in the first embodiment, a processing at Step S7 is performed. In the processing at Step S7, a determination is made as to whether the light source turned on in the processing at Step S11 included in Step S1 is the green LED 53. If the light source turned on in the processing at Step S11 is the green LED 53 (Yes at Step S7), the CPU 18 sets the weighting factors a and d to 0 and the weighting factors b and c to 1 (Step S8). If the light source turned on in the processing at Step S11 is not the green LED 53 (No at Step S7), the CPU 18 sets the weighting factors a and d to 1 and the weighting factors b and c to 0 (Step S9). After the processing at Step S8 or Step S9, the process starting at Step S2 described with reference to FIG. 7 in the first embodiment is performed.

Thus, in the second embodiment, if the light source turned on in the processing at Step S11 is the green LED 53, the weighting factors a and d is set to 0, and the weighting factors b and c is set to 1. That is, when detecting the green light, the outputs of the second and the third optical sensors 62 and 63 are employed, while the outputs of the first and the fourth optical sensors 61 and 64 are disabled. This is because, when detecting the pulsation by the artery 410 using the reflected light (reflected light 532) of the green light, the second and the third optical sensors 62 and 63 that are arranged closer than the first and the fourth optical sensors 61 and 64 to the green LED 53 are more advantageous in terms of the detection accuracy.

As illustrated in FIG. 2, among the first optical sensor 61, the second optical sensor 62, the third optical sensor 63, and the fourth optical sensor 64, the second optical sensor 62 is closest to and the fourth optical sensor 64 is farthest from the green LED 53. When the green LED 53 is on, the factor (b) corresponding to the output of the second optical sensor 62 (output PD2) is 1, and the factor (d) corresponding to the output of the fourth optical sensor 64 (output PD4) is 0. Therefore, among the factors (for example, a, b, c, and d) that are multiplied by the values corresponding to the outputs from the optical sensor 60 obtained when the green LED 53 is on, the factor (b) is larger than the factor (d). The factor (b) is a factor that is multiplied by a value corresponding to the output from the second optical sensor 62 that is closest to the green LED 53. The factor (d) is a factor that is multiplied by a value corresponding to the output from the fourth optical sensor 64 that is farthest from the green LED 53.

In the second embodiment, if the light source turned on in the processing at Step S11 is not the green LED 53, that is, the light source turned on is the red LED 51 or the near-infrared LED 52, the weighting factors a and d are set to 1, and the weighting factors b and c are set to 0. That is, when detecting the red light or the near-infrared light, the outputs of the first and the fourth optical sensors 61 and 64 are employed, while the outputs of the second and the third optical sensors 62 and 63 are disabled. This is because, when detecting the blood oxygen level using the red light or the near-infrared light scattered by blood in the artery 410, the first optical sensor 61 and the fourth optical sensor 64 that are arranged farther than the second and the third optical sensors 62 and 63 from the green LED 53 are more advantageous in terms of the detection accuracy.

As illustrated in FIG. 2, among the first optical sensor 61, the second optical sensor 62, the third optical sensor 63, and the fourth optical sensor 64, the third optical sensor 63 is closest to and the first optical sensor 61 is farthest from the near-infrared LED 52. Among the first optical sensor 61, the second optical sensor 62, the third optical sensor 63, and the fourth optical sensor 64, the second optical sensor 62 or the third optical sensor 63 is closest to and the first optical sensor 61 or the fourth optical sensor 64 is farthest from the red LED 51. When the red LED 51 or the near-infrared LED 52 is on, the factor (c) corresponding to the output of the third optical sensor 63 (output PD3) is 0, and the factor (a) corresponding to the output of the first optical sensor 61 (output PD1) is 1. Therefore, among the factors (for example, a, b, c, and d) that are multiplied by values corresponding to the outputs from the optical sensor 60 obtained when the red LED 51 or the near-infrared LED 52 is on, the factor (c) is smaller than the factor (a). The factor (c) is a factor that is multiplied by a value corresponding to the output from the third optical sensor 63 that is closest to the red LED 51 or the near-infrared LED 52. The factor (a) is a factor that is multiplied by a value corresponding to the output from the first optical sensor 61 that is farthest from the red LED 51 or the near-infrared LED 52.

The second embodiment is the same as the first embodiment except in the matters noted above. According to the second embodiment, the light source 50 includes a green light source (for example, green LED 53) and at least one of a red light source (for example, red LED 51) that emits the red light and an infrared light source (for example, near-infrared LED 52) that emits the infrared light. The optical sensors include two or more sensors (for example, first optical sensor 61, second optical sensor 62, third optical sensor 63, and fourth optical sensor 64) disposed at different distances from the green light source. Among the factors (for example, a, b, c, and d) that are multiplied by the values corresponding to the outputs from the optical sensors, the factor (b) is larger than the factor (d). The factor (b) is a factor that is multiplied by the value corresponding to the output from the optical sensor (for example, second optical sensor 62) that is closest to the green light source. The factor (d) is a factor that is multiplied by the value corresponding to the output from the optical sensor (for example, fourth optical sensor 64) that is farthest from the green light source. Therefore, in the detection of the light from the green light source, the optical sensor closest to the green light source can be more efficiently used. The intent of such use of the optical sensors is advantageous in, for example, the detection of reflected green light.

The optical sensors are four optical sensors (first optical sensor 61, second optical sensor 62, third optical sensor 63, and fourth optical sensor 64). The factors (b and c) that are multiplied by the value corresponding to the outputs from the two optical sensors (second optical sensor 62 and third optical sensor 63) that are relatively closer to the green light source (for example, green LED 53) are 1. The factors (a and d) that are multiplied by the value corresponding to the outputs from the optical sensors that are relatively farther from the green light source (first optical sensor 61 and fourth optical sensor 64) are 0. This configuration can more easily achieve the setting of the factors for more efficient use of the optical sensor that is closest to the green light source.

The light source 50 includes the green light source (for example, green LED 53) that emits green light, and at least one of the red light source (for example, red LED 51) that emits red light and the infrared light source (for example, near-infrared LED 52) that emits the infrared light. The optical sensors includes two or more sensors (for example, first optical sensor 61, second optical sensor 62, third optical sensor 63, and fourth optical sensor 64) disposed at different distances from the green light source. Among the factors that are multiplied by the values corresponding to the outputs from the optical sensors obtained when the one of the red light source and the infrared light source is on, the factor (for example, c) that is multiplied by the value corresponding to the output from the optical sensor (for example, third optical sensor 63) closest to the one light source is smaller than the factor (for example, a) that is multiplied by the value corresponding to the output from the optical sensor (for example, first optical sensor 61) farthest from the one light source. This configuration allows more efficient use of the optical sensor farthest from the one light source, in the detection of the light from the one light source. The intent of such use of optical sensors is advantageous in, for example, detection of scattering of the red light or the infrared light.

The optical sensors are the four optical sensors (first optical sensor 61, second optical sensor 62, third optical sensor 63, and fourth optical sensor 64). The factors (b and c) that are multiplied by the values corresponding to the outputs from the two optical sensors (for example, second optical sensor 62 and third optical sensor 63) that are relatively closer to at least one of the red light source (for example, red LED 51) that emits red light and the infrared light source (for example, near-infrared LED 52) that emits the infrared light are 0. The factors (a and d) that are multiplied by the values corresponding to the outputs from the optical sensors (the first optical sensor 61 and the fourth optical sensor 64) that are relatively farther from the one of the red light source and the infrared light source are 1. This configuration can more easily achieve the setting of the factors for more efficient use of the optical sensors that are farther from the one light source.

### Third Embodiment

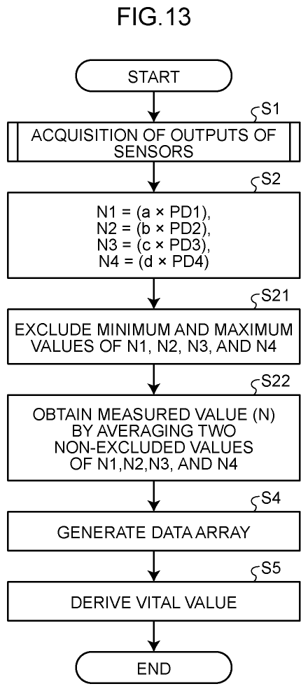

The following describes a third embodiment of the present disclosure in which processing partially different from that of the first and second embodiments is performed, with reference to FIG. 13.

FIG. 13 is a flowchart of a process for deriving the vital values performed in the third embodiment. In the third embodiment, processing at Step S21 and processing at Step S22 are performed instead of the processing at Step S3 in the first embodiment.

In the processing at Step S21, the CPU 18 excludes the minimum value and the maximum value from the values N1, N2, N3, and N4 calculated in the processing at Step S2. For example, if N1>N2>N3>N4 is satisfied as a magnitude relation between the values N1, N2, N3, and N4, the maximum value N1 and the minimum value N4 are excluded.

In the processing at Step S22, the measured value N is obtained by averaging two values that have not been excluded in the processing at Step S21. After the processing at Step S22, the process moves to Step S4. The measured value N that serves as a basis of the data array in the processing at Step S4 in the third embodiment is the measured value N obtained in the processing at Step S22.

The third embodiment is the same as the first embodiment except in the matters noted above. According to the third embodiment, the optical sensors includes four or more optical sensors (for example, first optical sensor 61, second optical sensor 62, third optical sensor 63, and fourth optical sensor 64), and the control circuit (for example, control circuit 10) calculates the average of the values excluding the maximum and minimum values from among four or more values corresponding to the outputs of the optical sensors and sets the calculated average as the measured value of the light measured by the optical sensors. Specifically, the control circuit obtains values excluding the maximum and minimum values from among four or more values corresponding to the outputs of the optical sensors, calculates the sum of the obtained values, divides the sum by a value (for example, 4−2=2) obtained by subtracting 2 from the total number of the optical sensors (for example, 4) to obtain an average, and sets the average as the measured value of the light measured by the optical sensors (for example, measured value N). As a result, even if at least one of the maximum and the minimum values is an outlier deviating from a value of an intended output of the optical sensor, the effect of the maximum and minimum values can be reduced. Therefore, the accuracy of detection can be more easily ensured.

### Modification of First and Third Embodiments

In first and third embodiments, the values of the weighting factors a and d may be larger than those of the weighting factors b and c. To give a specific example, the weighting factors may be set as a=d=2 and b=c=1. The first and the fourth optical sensors 61 and 64 are located farther from the light source 50 than the second and the third optical sensors 62 and 63 are. Therefore, the intensity of light detectable by the first and the fourth optical sensors 61 and 64 tends to be weaker than that by the second and the third optical sensors 62 and 63. Therefore, for the purpose of compensating such a tendency, the values of the weighting factors a and d may be set larger than the values of the weighting factors b and c.

Thus, the optical sensors include the two or more sensors (for example, first optical sensor 61, second optical sensor 62, third optical sensor 63, and fourth optical sensor 64) disposed at different distances from the light source (for example, light source 50). In this configuration, among the factors that are multiplied by the values corresponding to the outputs from the optical sensors obtained when the light source is on, a first factor may be set smaller than a second factor. The first factor is the factor (b or c) that is multiplied by the value corresponding to the output from the optical sensor closest to the light source (for example, second optical sensor 62 or third optical sensor 63). The second factor is the factor (a or d) that is multiplied by the value corresponding to the output from the optical sensor farthest from the light source (for example, first optical sensor 61 or fourth optical sensor 64). This configuration facilitates more even handling of the outputs of the respective sensors. That is, the factors facilitate even handling of the respective outputs of the sensors.

### Fourth Embodiment

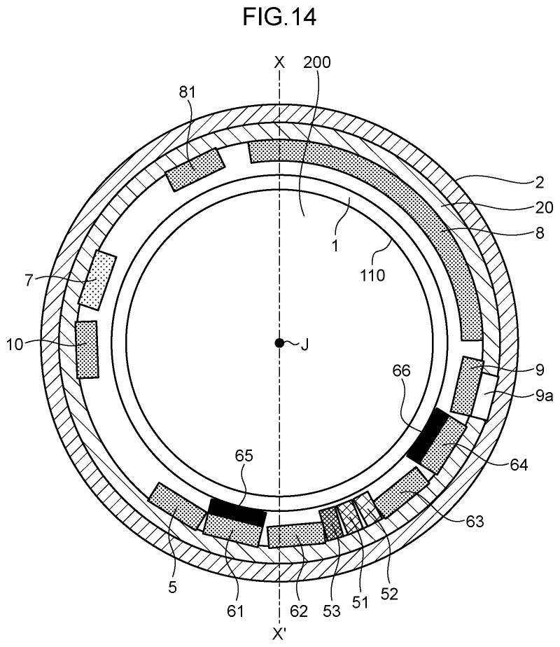

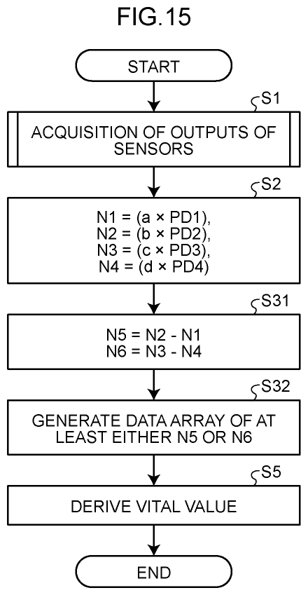

The following describes a fourth embodiment of the present disclosure in which processing partially different from that of the first, second, and third embodiments is performed, with reference to FIGS. 14 and 15.

FIG. 14 is a view illustrating an internal structure of the detection device employed in the fourth embodiment. As illustrated in FIG. 14, in the fourth embodiment, a light-blocking member 65 and a light-blocking member 66 are further provided in addition to the components of the detection device 100 of the first embodiment. The light-blocking member 65 and the light-blocking member 66 are light blockers. The light-blocking members 65 and 66 block the light emitted from the red LED 51, the near-infrared LED 52, and the green LED 53, and the reflected light and the scattered light of the emitted light. The light-blocking member 65 covers a light detection surface of the first optical sensor 61. The light-blocking member 66 covers a light detection surface of the fourth optical sensor 64.

Therefore, in the fourth embodiment, the second and the third optical sensors 62 and 63 each correspond to a first type optical sensor that is configured to detect the light emitted from the light source 50. The first and the fourth optical sensors 61 and 64 each correspond to a second type optical sensor covered with a light-blocking layer that blocks the light emitted from the light source 50.

FIG. 15 is a flowchart of a process for deriving the vital values performed in the fourth embodiment. In the fourth embodiment, processing at Step S31 and processing at Step S32 are performed instead of the processing at Step S3 and the processing at Step S4 in the first embodiment.

In the processing at Step S31, the CPU 18 calculates a difference value N5 obtained by subtracting the value N1 from the value N2. The CPU 18 also calculates a difference value N6 obtained by subtracting the value N4 from the value N3.

The difference values N5 and N6 each correspond to a value (difference value) obtained by subtracting a value corresponding to an output of the second type optical sensor from a value corresponding to an output of the first type optical sensor described above. The second and the first optical sensors 62 and 61 that are output sources of the values N2 and N1, respectively, that are the basis of calculation of the difference value N5 constitute a pair of the optical sensors that include the first type optical sensor and the second type optical sensor. The third and the fourth optical sensors 63 and 64 that are output sources of the values N3 and N4, respectively, that are the basis of calculation of the difference value N6 constitute a pair of the optical sensors that include the first type optical sensor and the second type optical sensor. Thus, in the fourth embodiment, two pairs of the optical sensors are provided, each pair including the first type optical sensor and the second type optical sensor.

In the processing at Step S32, the CPU 18 generates at least one of a data array in which the difference values N5 are arranged in chronological order and a data array in which the difference values N6 are arranged in chronological order. The processing at Step S32 is the same as the processing at Step S4 except in that the data in chronological order is not the measured values N, but at least either of the difference values N5 and N6.

In the fourth embodiment, the light-blocking member 65 covers the light detection surface of the first optical sensor 61, so that the light from the light source 50 is not detected by the first optical sensor 61. That is, the output of the first optical sensor 61 is an output when no light is detected. This means that the output when an optical sensor such as the first optical sensor 61 or the second optical sensor 62 cannot detect light can be obtained from the first optical sensor 61 at any time regardless of the state of lighting of the light source 50. Assume that the term “reference output” refers to the output when the optical sensor cannot detect light, the first optical sensor 61 is a sensor from which the reference output is obtained. For the same reason, the fourth optical sensor 64 provided with the light-blocking member 66 is also a sensor from which the reference output is obtained.

In the processing at Step S31, the difference value N5 is the value obtained by subtracting the value N1 from the value N2. The values N2 and N1 are the values calculated in the processing at Step S2. The value N2 is a value corresponding to the output PD2, that is, the output of the second optical sensor 62 obtained in the processing at Step S13. The value N1 is the value corresponding to the output PD1, that is, the output of the first optical sensor 61 obtained in the processing at Step S12. As described above, the first optical sensor 61 is the sensor from which the reference output is obtained. Thus, the difference value N5 obtained by subtracting the value N1 from the value N2 corresponds to a value obtained by subtracting the value corresponding to the reference output from the value corresponding to the output of the second optical sensor 62. This means that the difference value N5 can be calculated as a value obtained by subtracting “the output when the optical sensor cannot detect light” from “the output of the optical sensor that detects light when present and produces an output corresponding to the intensity of the detected light”. For example, assume that a first case is a case where the second optical sensor 62 cannot detect any light at all. The output of the second optical sensor 62 in the first case should be substantially the same as the output of the first optical sensor 61 that cannot detect light regardless of the presence or absence of light. Therefore, the difference value N5 in the first case should be substantially 0. In contrast, assume that a second case is a case where the second optical sensor 62 detects light. The output of the second optical sensor 62 in the second case should be significantly different from the output of the first optical sensor 61 that cannot detect light regardless of the presence or absence of light. Thus, the difference value N5 in the second case is significantly larger than 0. Thus, by shielding the first optical sensor 61 from light with the light-blocking member 65 and calculating the difference value N5, it is possible to more accurately determine whether the optical sensor has detected at least a slight amount of light or no light at all. Similarly, by shielding the fourth optical sensor 64 from light with the light-blocking member 66 and calculating the difference value N6, it is possible to more accurately determine whether the optical sensor has detected at least a slight amount of light or no light at all.

The fourth embodiment is the same as the first embodiment except in the matters noted above. According to the fourth embodiment, the optical sensors includes the first type optical sensors (for example, second optical sensor 62 and third optical sensor 63) configured to detect light emitted from the light source (for example, light source 50) and the second type optical sensors (for example, first optical sensor 61 and fourth optical sensor 64) covered with the light-blocking layers (for example, light-blocking members 65 and 66) that block the light emitted from the light source. The control circuit (for example, control circuit 10) calculates the measured values (for example, difference values N5 and N6) of the light measured by the optical sensors based on the values obtained by subtracting the values corresponding to the outputs of the second type optical sensors from the values corresponding to the outputs of the first type optical sensors. With this configuration, whether the first type optical sensors have detected at least a slight amount of light or no light at all can be more accurately determined.

The following describes modifications of the fourth embodiment in which the processing at Step S32 in the fourth embodiment is replaced with more specific processes.

### First Modification of Fourth Embodiment

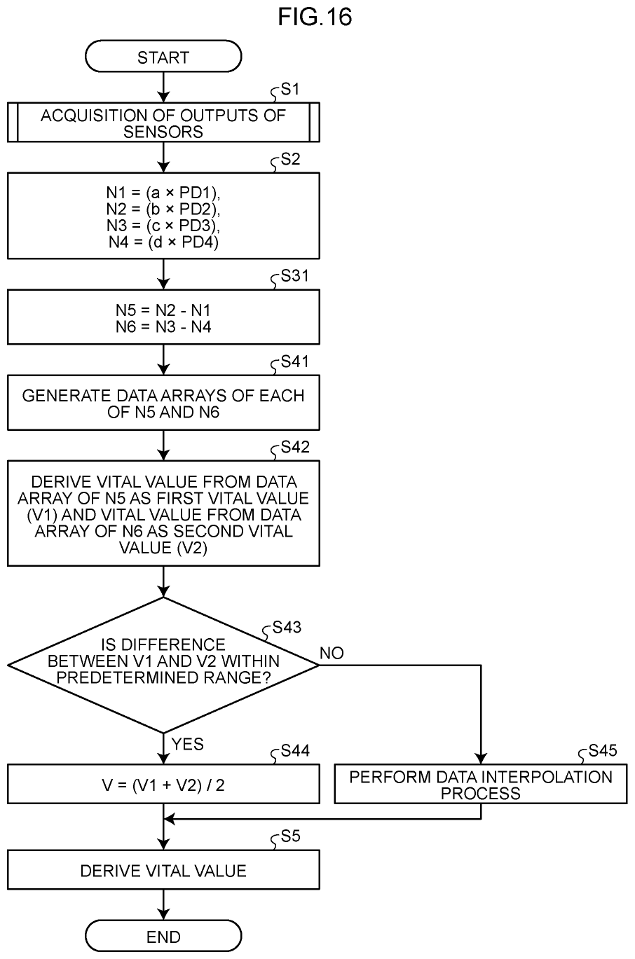

FIG. 16 is a flowchart of a process for deriving the vital values performed in a first modification of the fourth embodiment. In the first modification of the fourth embodiment, Step S41, Step S42, Step S43, Step S44, and Step S45 are performed instead of Step S32 in the fourth embodiment.

In the processing at Step S41, the CPU 18 generates both the data array in which the difference values N5 are arranged in chronological order and the data array in which the difference values N6 are arranged in chronological order. In other words, while the processing at Step S31 in the fourth embodiment generates at least either the data array in which the difference values N5 are arranged in chronological order or the data array in which the difference values N6 are arranged in chronological order, the processing at Step S41 in the first modification of the fourth embodiment always generates both of them.

In the processing at Step S42, the CPU 18 derives a vital value from the data array of the difference values N5 and a vital value from the data array of the difference values N6, the difference values N5 and N6 having been generated in the processing at Step S41. That is, the same processing as that at Step S5 is performed on each of the data array of the difference value N5 and the data array of the difference value N6. The CPU 18 sets the vital value derived from the data array of the difference values N5 as a vital value V1 and the vital value derived from the data array of the difference values N6 as a vital value V2.

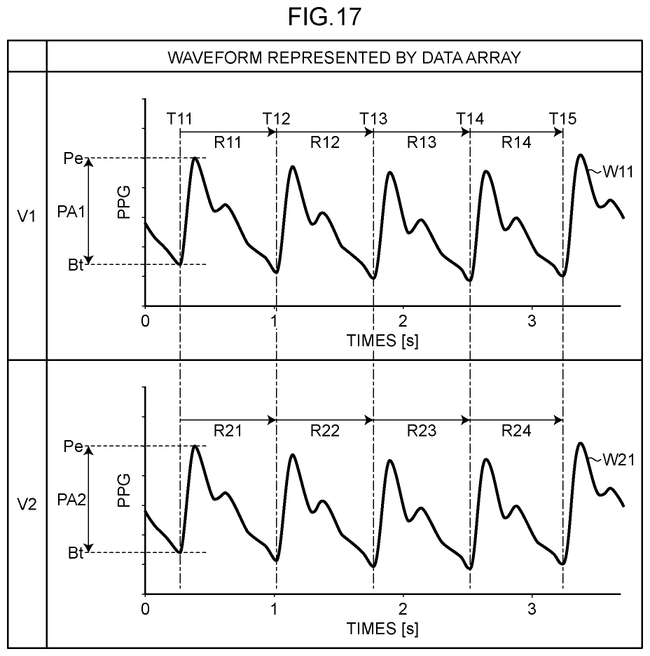

In the processing at Step S43, a determination is made as to whether the difference between the vital values V1 and V2 derived in the processing at Step S42 is within a predetermined range. The concept of the difference between the vital values V1 and V2 will be described below with reference to FIG. 17.

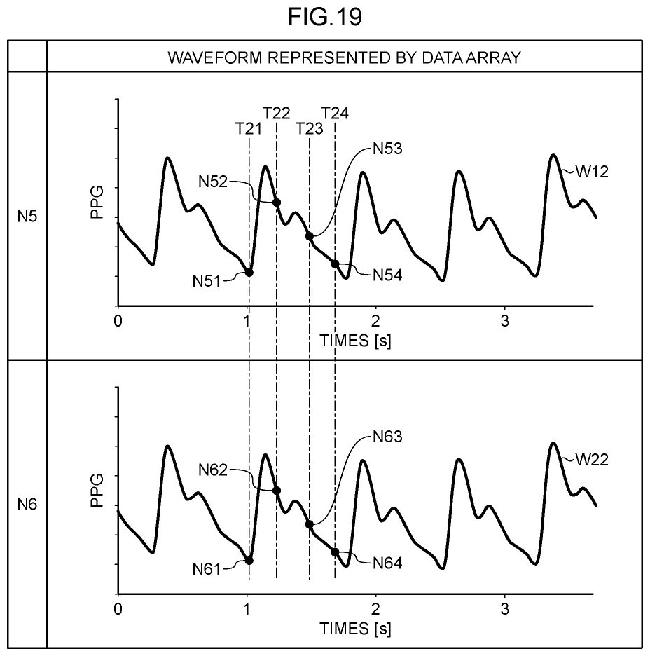

FIG. 17 illustrates diagrams illustrating the concept of the difference between the vital values V1 and V2. FIG. 17 and FIG. 19 to be described later illustrate the concept when the vital values are values indicating the pulsation. When the values indicating the pulsation is derived as the vital values, the data array from which the vital values are derived simultaneously indicates a time length and a wave height in the pulsation.

The time length herein refers to the length of time from the start of one pulse to the end of the pulse. The time when the pulse ends is the time when the next pulse starts. For example, in FIG. 17, the pulse for the vital value V1 starting at time T11 ends at time T12, and the time length of the pulse is time length R11. The pulse for the vital value V2 starting at time T11 also ends at time T12, and the time length of the pulse is time length R21. The pulse for the vital value V1 starting at time T12 ends at time T13, and the time length of the pulse is time length R12. The pulse for the vital value V2 starting at time T12 also ends at time T13, and the time length of the pulse is time length R22. The pulse for the vital value V1 starting at time T13 ends at time T14, and the time length of the pulse is time length R13. The pulse for the vital value V2 starting at time T13 also ends at time T14, and the time length of the pulse is time length R23. The pulse for the vital value V1 starting at time T14 ends at time T15, and the time length of the pulse is time length R14. The pulse for the vital value V2 starting at time T14 also ends at time T15, and the time length of the pulse is time length R24.

The wave height herein corresponds to a difference in the sensor output due to repetitive alternation in intensity of the reflected light 532. Specifically, the wave height is caused corresponding to repetitive alternation between the non-dilated portion 411 and the dilated portion 412 of the artery 410 (refer to FIG. 9) in the irradiation area 530. FIG. 17 individually illustrates the wave heights in the pulsation between time T11 and time T12 as a wave height PA1 of the vital value V1 and a wave height PA2 of the vital value V2. The wave height PA1 is the difference between a peak Pe and a bottom Bt in the pulsation of the vital value V1 that occurs in the time length R11 from time T11 to time T12. The wave height PA2 is the difference between the peak Pe and the bottom Bt in the pulsation of the vital value V2 that occurs in the time length R21 from time T11 to time T12. FIG. 17 illustrates the wave heights only for the pulsation that occurs between time T11 and time T12, but the wave heights for other pulsations can also be identified from the data array.

The CPU 18 compares the time length and the wave height of the pulsation indicated by the vital value V1 with the time length and the wave height of the pulsation indicated by the vital value V2, and determines whether the difference therebetween falls within a predetermined degree. To give a specific example, the CPU 18 calculates the average of the time lengths and the average of the wave heights for a plurality of the pulsations that have occurred within a certain period of time separately for each of the vital values V1 and V2.

The CPU 18 compares the average of the time lengths of the pulses of the vital value V1 (first average) with the average of the time lengths of the pulses of the vital value V2 (second average). The CPU 18 determines whether, when one of the first and the second averages is assumed to be a value of 100%, the other is a value that falls within a range of “the one average±a predetermined proportion (for example, ±10%) thereof”. If the other falls within the range, the CPU 18 determines that the difference between the vital values V1 and V2 falls within the predetermined degree.

The CPU 18 also compares the average of the wave heights of the pulsations of the vital values V1 (third average) with the average of the wave heights of the pulsations of the vital values V2 (fourth average). The CPU 18 determines whether, when one of the third and the fourth averages is assumed to be a value of 100%, the other is a value that falls within a range of “the one average±a predetermined proportion (for example, ±10%) thereof”. If the other falls within the range, the CPU 18 determines that the difference between the vital values V1 and V2 falls within the predetermined degree. If the CPU 18 determines that the difference between the vital values V1 and V2 for both the time length and the wave height falls within the predetermined degree, the CPU 18 determines that the difference between the vital values V1 and V2 is within the predetermined range in the processing at Step S43 (Yes at Step S43). Otherwise, the CPU 18 determines that the difference between the vital values V1 and V2 is not within the predetermined range (No at Step S43).

In the example described above, the average calculated from the vital values V1 is compared with the average calculated from the vital values V2 after calculating the average for a plurality of pulsations, such as the first average, the second average, the third average, and the fourth average. The method of comparison is, however, not limited thereto. For example, instead of calculating the averages, the time lengths and the wave heights of the pulsations that occur substantially at the same time among the pulsations indicated by the vital values V1 and the pulsations indicated by the vital values V2 may be individually compared. In this case, the time lengths and the wave heights may be compared for sampled some of multiple pulsations, or the time lengths and the wave heights may be compared for all the multiple pulsations. When the following condition is satisfied, the difference between the vital values V1 and V2 may be determined to fall within the predetermined degree. The condition is a condition related to a comparison between the time length (or wave height) of the pulsation indicated by the vital value V1 and the time length (or wave height) of the pulsation indicated by the vital value V2. The condition is determined to be satisfied when the proportion of predetermined pulsations among all pulsations subjected to the comparison is a predetermined proportion or greater (for example, 70% or greater). Here, the predetermined pulsation denotes a pulsation in which, when one of the time lengths (or wave height) of the vital values V1 and V2 is assumed to be a value of 100%, the other falls within a range of “the one time length (or wave height)±a predetermined proportion (for example, ±10%) thereof”. The “all pulsations subjected to the comparison” denotes all of the plurality of pulsations obtained by obtaining the sensor outputs the plurality of times within the certain period of time, which are indicated by the data array (refer to Step S1 to Step S4 in FIG. 8).

If, in the processing at Step S43, the difference between the vital values V1 and V2 is determined to be within the predetermined range (Yes at Step S43), the CPU 18 calculates the average of the vital values V1 and V2 ((V1+V2)/2) as a vital value V (Step S44). Referring to FIG. 17 for explanation, the vital value V has a waveform of an intermediate data array between the vital value V1 and the vital value V2. If the vital values V1 and V2 completely match each other, the vital value V is the same as the vital values V1 and V2.

If, in the processing at Step S43, the difference between the vital values V1 and V2 is determined not to be within the predetermined range (No at Step S43), a data interpolation process is performed (Step S45). A specific interpolation method in the data interpolation process is as follows. A predicted value is calculated based on data at two or more time points (points) in the past, and, if the difference between the predicted value and the average of the vital values V1 and V2 is within a predetermined range, the predicted value is employed. If the difference is outside the predetermined range, one of the vital values V1 and V2 that is closer to the predicted value is selected. In the case of employing an operation based on linear approximation as an example of a specific method of calculating the predicted value, if the vital values V are y1 and y2 at two latest time points (t1 and t2), and the vital value V is y3 at a time point (t3) when the predicted value is applied, y3 is calculated as given in Expression (1) below.

$$y 3 = ( ( y 2 - y 1 ) / ( t 2 - t 1 ) ) ( t 3 - t 1 ) + y 1 \qquad ( 1 )$$

In the first modification of the fourth embodiment, after the processing at Step S44 or the processing at Step S45, the process moves to Step S5. The first modification of the fourth embodiment is the same as the fourth embodiment except in the matters noted above.

### Second Modification of Fourth Embodiment

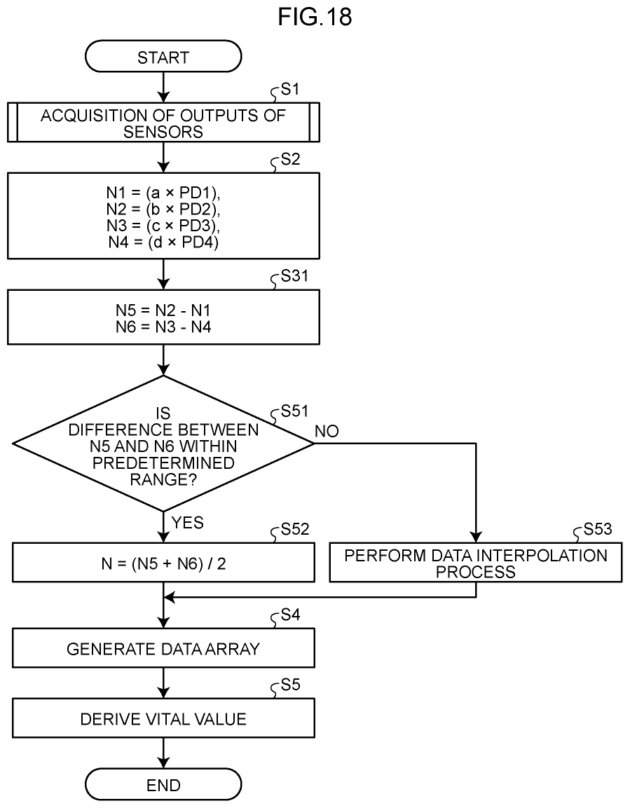

FIG. 18 is a flowchart of a process for deriving the vital values performed in a second modification of the fourth embodiment. In the second modification of the fourth embodiment, Step S51, Step S52 or Step S53, and Step S4 described with reference to FIG. 7 in the first embodiment are performed instead of Step S32 in the fourth embodiment.

In the processing at Step S51, a determination is made as to whether the difference between the difference values N5 and N6 calculated in the processing at Step S31 is within a predetermined range. The concept of the difference between the difference values N5 and N6 will be described below with reference to FIG. 19.

FIG. 19 illustrates diagrams illustrating the concept of the difference between the difference values N5 and N6. As described with reference to FIG. 17 in the first modification of the fourth embodiment, the vital values V1 and V2 that are the data arrays of the difference values N5 and N6, respectively, can be depicted as waveforms indicating the pulsations. Thus, the difference values N5 and N6 can be regarded as a “part of a data set that can be represented as a waveform indicating a pulsation by being arranged as a data array in chronological order”.

In the second modification of the fourth embodiment, the CPU 18 compares the difference values N5 and N6 calculated based on the output of optical sensor 60 at a certain time. That is, as for the difference values N5 and N6 compared in the processing at Step S51, the outputs PD1 and PD2 for the difference value N5 and the outputs PD3 and PD4 for the difference value N6 are outputs at the same time.

FIG. 19 illustrates times T21, T22, T23, and T24 as examples of times compared in the processing at Step S51. In the case of the example illustrated in FIG. 19, the CPU 18 compares a difference value N51 with a difference value N61. The difference value N51 is the value of the difference value N5 at time T21, and the difference value N61 is the value of the difference value N6 at time T21. The CPU 18 compares a difference value N52 with a difference value N62. The difference value N52 is the value of the difference value N5 at time T22, and the difference value N62 is the value of the difference value N6 at time T22. The CPU 18 compares a difference value N53 with a difference value N63. The difference value N53 is the value of the difference value N5 at time T23, and the difference value N63 is the value of the difference value N6 at time T23. The CPU 18 compares a difference value N54 with a difference value N64. The difference value N54 is the value of the difference value N5 at time T24, and the difference value N64 is the value of the difference value N6 at time T24.

The CPU 18 determines whether, when one of the difference values N5 and N6 at a certain time subjected to comparison is assumed to be a value of 100%, the other is a value that falls within a range of “the one difference value±a predetermined proportion (for example, ±10%) thereof”. If the other falls within the range, the CPU 18 determines that the difference between the difference values N5 and N6 at the certain time falls within the predetermined degree. If a predetermined condition is satisfied, the CPU 18 determines that the difference between the difference values N5 and N6 is within the predetermined range (Yes at Step S51). The predetermined condition is that, among values of the difference values N5 and N6 compared within a certain period of time during which the acquisition of the output of the optical sensor 60 is continued in the derivation of the value indicating the pulsation, values for which the difference between the difference values N5 and N6 is determined to fall within the predetermined degree occupy a predetermined proportion or greater (for example, 70% or greater) of all the values compared. The “all the values compared” herein may include all the results of comparison between the difference values N5 and N6 obtained within the certain period of time, or may be constituted by the results of comparison between the difference values N5 and N6 obtained at some times, which are sampled within a certain period of time, such as the times T21, T22, T23, and T24 described with reference to FIG. 19.

If, in the processing at Step S51, the difference between the difference values N5 and N6 is determined to be within the predetermined range (Yes at Step S51), the CPU 18 calculates the average of the difference values N5 and N6 ((N5+N6)/2) as the measured value N (Step S52). If, in the processing at Step S51, the difference between the difference values N5 and N6 is determined not to be within the predetermined range (No at Step S51), the data interpolation process is performed (Step S53). A specific interpolation method in the data interpolation process is as follows. A predicted value is calculated based on data at two or more time points (points) in the past. If the difference between the predicted value and the average of the difference values N5 and N6 is within a predetermined range, the predicted value is employed. If the difference is outside the predetermined range, a difference value that is closer to the predicted value is selected from the difference values N5 and N6. When an operation based on the linear approximation is employed as an example of a specific method of calculating the predicted value, Expression (2) below is used. In Expression (2), y4 and y5 denote the measured values N at two latest time points (t4 and t5), respectively. y6 denotes the measured value N at a time point (t6) when the predicted value is applied.

$$y 6 = ( ( y 5 - y 4 ) / ( t 5 - t 4 ) ) ( t 6 - t 4 ) + y 1 \qquad ( 2 )$$

In the second modification of the fourth embodiment, after the processing at Step S52 or the processing at Step S53, the process moves to Step S4. The measured value N in the processing performed at Step S4 is the measured value N calculated in the processing at Step S52 or S53. After the processing at Step S4, the process moves to Step S5 in the same way as in the first embodiment. The second modification of the fourth embodiment is the same as the fourth embodiment except in the matters noted above.

According to the fourth embodiment and the modifications thereof, the above-described two pairs of the optical sensors are provided, each pair including the first type optical sensor and the second type optical sensor. With this configuration, the accuracy of detection using the output of each of the two pairs of the optical sensors can be ensured.

The control circuit (control circuit 10) calculates the difference value (difference value N5 or N6) obtained by subtracting the value corresponding to the output of the second type optical sensor from the value corresponding to the output of the first type optical sensor for each of the two pairs of the optical sensors. If the difference between the difference value (difference value N5) of one of the two pairs of the optical sensors and the difference value (difference value N6) of the other of the two pairs of the optical sensors is within the predetermined range, the average of the difference values of the two pairs of the optical sensors is calculated as the measured value of the light measured by the optical sensors. Thereby, it is possible to more accurately determine whether the first type optical sensors has detected at least a slight amount of light or no light at all. Furthermore, the certainty of the outputs of the two pairs of the optical sensors can be ensured by comparing the difference values corresponding to the outputs of each of the two pairs of the optical sensors. Thus, the accuracy of detection can be ensured more reliably.

The identification of the blood oxygen level does not require both the red light and the near-infrared light, but requires only one of them. Therefore, at least one of the red LED 51 and the near-infrared LED 52 of the light source 50 needs to be included in the detection device 100, and the other need not be included. However, in each of the embodiments described above, both the red LED 51 and the near-infrared LED 52 are provided and used for the purpose of more accurate identification of the blood oxygen level.

The number of the optical sensors is not limited to four. For example, the number of the optical sensors may be two, such as only the first and second optical sensors 61 and 62, or only the third and fourth optical sensors 63 and 64. The number of the optical sensors may be three or five or more. The factors of the optical sensors are not limited to the four factors a, b, c, and d, and are provided individually for the optical sensors provided in the detection device. Thus, if the optical sensors are, for example, only the first and the second optical sensors 61 and 62, only a and b are sufficient as the factors.

The light source 50 is not limited to the LEDs. The light source 50 may be organic light-emitting diodes (OLEDs), for example. Otherwise, the light source 50 may be configured by other light-emitting elements that function in the same way as the red LED 51, the near-infrared LED 52, and the green LED 53 of the embodiments.

In the embodiments and the modifications thereof, the inner surface portion 1 is a light-transmitting member, so that the light from the light source 50 can be emitted toward the hollow portion 200 and the light can be detected by the optical sensor 60. However, these features may be achieved by other methods. For example, the detection device may employ a member that is provided with openings at locations corresponding to the light source 50 and the optical sensor 60 on the inner peripheral surface of the finger ring-shaped housing and makes up the non-light-transmitting inner peripheral surface.

Other operational advantages accruing from the aspects described in the embodiments of the present disclosure that are obvious from the description herein, or that are conceivable as appropriate by those skilled in the art will naturally be understood as accruing from the present disclosure.

What is claimed is:

1. A detection device comprising:
a light source;
a plurality of optical sensors; and
a control circuit configured to calculate values based on outputs of the optical sensors, wherein
the control circuit is configured to perform processing to multiply values corresponding the outputs of the optical sensors by factors individually for each of the values corresponding to the outputs the optical sensors.

2. The detection device according to claim 1, wherein
the light source comprises:
a green light source configured to emit green light; and
at least one of a red light source configured to emit red light or an infrared light source configured to emit infrared light,
the optical sensors comprise two or more sensors disposed at different distances from the green light source, and
among the factors to be multiplied by values corresponding to the outputs from the optical sensors obtained when the green light source is on, a factor to be multiplied by a value corresponding to an output from an optical sensor closest to the green light source is larger than a factor to be multiplied by a value corresponding to an output from an optical sensor farthest from the green light source.

3. The detection device according to claim 2, wherein
the optical sensors are four optical sensors, and
factors to be multiplied by values corresponding to outputs from two of the optical sensors relatively closer to the green light are 1, and a factor to be multiplied by a value corresponding to an output from an optical sensor relatively farther from the green light is 0.

4. The detection device according to claim 1, wherein
the light source comprises:
a green light source configured to emit green light; and
at least one of a red light source configured to emit red light or an infrared light source configured to emit infrared light,
the optical sensors comprise two or more sensors disposed at different distances from the one of the red light source and the infrared light source, and
among the factors to be multiplied by values corresponding to the outputs from the optical sensors obtained when the one light source is on, a factor to be multiplied by a value corresponding to an output from an optical sensor closest to the one light source is smaller than a factor to be multiplied by a value corresponding to an output from an optical sensor farthest from the one light source.

5. The detection device according to claim 4, wherein
the optical sensors are four optical sensors, and
factors to be multiplied by values corresponding to outputs from two of the optical sensors relatively closer to the one light source are 0, and a factor to be multiplied by a value corresponding to an output from an optical sensor relatively farther from the one light source is 1.

6. The detection device according to claim 1, wherein
the optical sensors comprise two or more sensors disposed at different distances from the light source, and
among the factors to be multiplied by values corresponding to the outputs from the optical sensors obtained when the light source is on, a factor to be multiplied by a value corresponding to an output from an optical sensor closest to the light source is smaller than a factor to be multiplied by a value corresponding to an output from an optical sensor farthest from the light source.

7. The detection device according to claim 1, wherein
the optical sensors comprise four or more optical sensors, and
the control circuit is configured to calculate an average obtained by dividing a sum of values excluding maximum and minimum values among four or more values corresponding to the outputs of the optical sensors by a value obtained by subtracting two from the number of the optical sensors as a measured value of light measured by the optical sensors.

8. The detection device according to claim 1, wherein
the optical sensors comprise a first optical sensor configured to detect light emitted from the light source and a second optical sensor covered with a light-blocking layer configured to block the light emitted from the light source, and
the control circuit is configured to calculate measured values of light measured by the optical sensors based on a value obtained by subtracting a value corresponding to an output of the second optical sensor from a value corresponding to an output of the first optical sensor.

9. The detection device according to claim 8, provided with two pairs of optical sensors, each pair comprising the first optical sensor and the second optical sensor.

10. The detection device according to claim 9, wherein
the control circuit is configured to:
calculate a difference value obtained by subtracting a value corresponding to the output of the second optical sensor from a value corresponding to the output of the first optical sensor for each of the two pairs of the optical sensors, and
if a difference between the difference value of one of the two pairs of the optical sensors and the difference value of the other of the two pairs of the optical sensors is within a predetermined range, calculate an average of the difference values of the two pairs of the optical sensors as a measured value of the light measured by the optical sensors.

11. The detection device according to claim 1, wherein the optical sensors are organic photodiodes.

12. The detection device according to claim 1, wherein the light source, the optical sensors, and the control circuit are accommodated in a finger ring-shaped housing.
